(19) United States
(12) Patent Application Publication
(10) Pub. No.: US 2018/0168469 A1
(43) Pub. Date: Jun. 21, 2018

(54) DEVICE AND METHOD FOR MEASURING PRESSURE IN A PATIENT'S HEART

(71) Applicant: Berlin Heart GmbH, Berlin (DE)

(72) Inventor: Marcus Granegger, Zürich (CH)

(73) Assignee: Berlin Heart GmbH, Berlin (DE)

(21) Appl. No.: 15/738,486

(22) PCT Filed: Jun. 16, 2016
(86) PCT No.: PCT/EP2016/063960

§ 371 Date: Dec. 20, 2017

(30) Foreign Application Priority Data
Jun. 22, 2015 (EP) 15173069.4

(51) Int. Cl. A61B 5/0215; A61B 5/00; A61M 1/10; A61B 5/07

(52) U.S. Cl. CPC A61B 5/02158; A61B 5/686; A61M 1/1086; A61B 5/076; A61M 2230/30; A61B 2562/0247; A61B 2562/168; A61B 2560/0219; A61M 2205/3331; A61B 5/4836

(57) ABSTRACT

A device for measuring pressure in a patient's heart is provided that comprises a sensor device, which detects the difference in pressure between a point inside the heart and the space lying outside the heart inside the thorax. An element of the sensor device, such as a sensor, may be arranged on a device connected to the heart, such as a heart pump or a cardiac pacemaker.

### CROSS-REFERENCE TO RELATED APPLICATIONS

This application is a 371 nationalization of international patent application PCT/EP2016/063960 filed Jun. 16, 2016, the entire contents of which are hereby incorporated by reference, which in turn claims priority under 35 USC § 119 to European patent application 15173069.4 filed Jun. 22, 2015.

### TECHNICAL FIELD

The invention lies in the field of mechanical and electrical engineering and can be used particularly advantageously in the field of medical technology. Specifically, the invention is concerned with the measuring of pressure in a patient's heart with the aid of sensor devices.

### BACKGROUND

A large number of devices have been developed within the medical field which are used to monitor or support patients and which can be implanted fully or partially in the patient's body. These include, for example, cardiac pacemakers, defibrillators, and also heart support systems in the form of fully or partially implanted heart pumps.

For some implantable devices of this kind, the monitoring of the fluid pressure within the heart, for example in a ventricle or atrium, is important.

For a physical and physiological analysis of such measurement data, a comparison with reference pressures or the consideration of a reference pressure is indispensable. For purposes such as these, absolute pressure sensors have thus been used in the heart in conjunction with a measurement of the barometric pressure outside the body. Examples of such pressure measurements are known from documents US 2007/0282210 A1, EP 1 415 672 B1 and U.S. Pat. No. 6,328,699 B1. Document WO 2013/122459 A1 describes, independently of the operation of a device, a diagnosis system which measures the pressure difference between the right intra-atrial pressure and the pericardium so as to identify a cardiac tamponade.

### BRIEF DESCRIPTION OF THE DRAWINGS

FIG. 1 shows a patient's body in a schematic illustration, showing a heart and a heart pump;

FIG. 2 shows a schematic illustration of a heart as a cavity with a heart pump connected thereto with sensors;

FIG. 3 schematically shows an illustration of a heart with two sensors, which are connected to a device;

FIG. 4 shows a schematic illustration of a heart with two possible arrangements of devices which each have sensors for measuring pressure;

FIG. 5 shows the structure of an axial/radial pump with a differential pressure sensor; and

FIG. 6 shows the structure of an axial pump with a differential pressure sensor.

### DETAILED DESCRIPTION

The object of the present invention, against the background of the prior art, is to create a device for measuring pressure in a patient's heart, which device makes it possible to measure a corrected pressure in conjunction with the operation of a device connected to the patient's heart, moreover in an efficient, most simple and economical manner, which also causes minimal stress for the patient.

The object is thus achieved by a device for measuring pressure in a patient's heart, characterised by a sensor device which detects the difference in pressure between a point inside the heart on the one hand and the space lying outside the heart inside the thorax on the other hand. Here, at least one element of the sensor device can be arranged on a device connected to the heart, in particular a heart pump, a cardiac pacemaker or defibrillator.

The pressure inside a heart is influenced both by the actual ventricle pressure, given by the static and dynamic conditions in the heart itself, and by the pressure prevailing outside the heart in the thorax and the barometric pressure outside the patient's body and acting thereon.

In order to determine certain physiological variables in conjunction with the monitoring of a heart, often only the difference between the absolute pressure inside the heart and the pericardium or thorax is important, which is also referred to as the transmural pressure and which is responsible for the forces acting on the heart wall. It is therefore almost impossible to identify the prevailing conditions exclusively on the basis of a pressure measurement inside the heart. The consideration of the barometric pressure alone is also of no help, since the thoracic pressure varies periodically, for example when breathing in and out, and therefore so too does the pressure inside the heart. For example, in the case of an implanted heart pump, which draws in blood by means of a heart catheter inside the heart, it is not possible to draw a clear conclusion in relation to the aspiration of the cannula on a heart wall merely by means of a measurement of the pressure within the heart (ventricle or atrium), since pressure fluctuations can also be caused by pressure fluctuations in the thorax. The transmural pressure is less affected by effects of this kind.

The present solution allows the measurement of the transmural pressure and therefore an improved possibility for controlling the particular device connected to the heart, for example a heart pump, a cardiac pacemaker or a defibrillator.

The sensor device can be connected for this purpose both to the space inside the heart/the heart wall and to the space outside the heart in the pericardium or in the thorax. Here, as will be explained in greater detail further below, the connection can be provided electrically or hydraulically (by means of a fluid channel) or by means of another type of communication. In any case, the connection of at least part of the sensor device to a device is advantageously such that the outlay for production and also for implantation of the overall assembly is optimised. Error sources during operation can thus be minimised, and a reliable placement in the patient's body can be achieved.

At least one sensor of the sensor device and as applicable also one fluid channel, to which the sensor is connected or in which the sensor is arranged, can be fixedly connected to a device, for example a heart pump, or can be secured thereto.

For example, one embodiment of the solution can provide that at least one element of the sensor device is arranged on a device connected to the heart, in particular a heart pump. In this case it can be provided that the sensor device has just a single pressure sensor in the form of a differential pressure sensor. This sensor for example can have a measuring membrane, wherein the pressure from inside the heart is present on a first side of the membrane, whereas the pressure from the thorax region is present on the second side of the membrane. For example, a membrane or sensor device of this type can directly close a passage opening in the heart wall or can be provided within the heart wall. However, a differential pressure sensor of this kind can also be connected on one or both sides of the membrane to the interior of the heart on the one hand and to the pericardium or the thorax region on the other hand, in each case by means of a fluid channel, so that the two pressures to be compared act on the element for measuring a pressure difference, in particular the membrane. The corresponding fluid channels can be formed by fixed tubular elements or cannulas or catheters. They can also be formed by bores and/or channels within a solid body, for example a housing of a device, for example in or on a heart pump.

A further embodiment of the solution for example can also provide that the differential pressure sensor on the one hand is connected to a region within the pump or in the intake region thereof or delivery region thereof and on the other hand is connected to a region outside the pump housing. A measurement of this kind in principle means that a difference in pressure between the interior of the pump, either in the intake or delivery region, on the one hand and outside the pump in the thorax on the other hand is measured. The pressure difference between the intake and delivery region created by the operation of the pump can be taken into consideration when determining the pressure in the blood vessels and in the heart. This thus results in a particularly favourable arrangement of all channels on a pump of this kind or in the region of the pump in the interior thereof and on the outside thereof, so that the entire assembly, formed of the sensor device and a heart pump of this kind, can be produced, implanted and operated particularly favourably.

A further embodiment of the solution can provide that the channel runs through the interior of a pump housing and is surrounded at least in part, in particular in portions, on all sides by the blood conveyed through the pump. If a channel of this kind runs within a pump, the assembly is particularly space-saving and less susceptible. Here, however, problems with regard to the spatial requirement within the pump housing need to be solved, because for example moving parts there require space and must not be hindered by the channel. Particular problems can arise for example with the use of axially conveying rotor pumps, since in pumps of this kind the majority of the interior of the pump housing is swept through as the conveying rotor rotates.

Good solution possibilities are provided for example with the use of rotors of this kind which have a cavity in their rotation axis region or within a hub, in which cavity a fluid channel of this kind can be arranged. A pump of this kind for example can also draw in blood in the axial direction and convey it in the radial direction. On the other hand, a fluid channel of this kind can also run in or on a pump on the housing wall separately from the interior of the pump or can be formed as a bore in the wall of the pump housing.

The solution can also be embodied in that the sensor device has two absolute pressure sensors, the first of which is arranged within the heart or is connected to the interior of the heart by means of a fluid channel and the second of which is arranged outside the heart in the thorax or is connected to a region outside the heart in the thorax by means of a fluid channel, and these are connected to a unit for determining a difference in pressure. In this case, depending on the arrangement of the device, in particular the heart pump, at least one of the absolute pressure sensors can be secured to the housing of the device and can measure the pressure outside or inside the housing. In the case of a heart pump, for example with part of the pump housing arranged outside the heart, an absolute pressure sensor can be arranged on the outside of the housing and can also be secured thereto as appropriate and can communicate with the pericardium or the thorax outside the pericardium directly or by means of a fluid channel. An absolute pressure sensor of this kind can also be arranged within the pump housing and can communicate by means of a channel running through the wall of the pump housing with the corresponding regions outside the heart. An absolute pressure sensor can also be fixedly arranged within the heart on the outside or inside of the pump housing and can be connected to the housing.

It can then be provided that the device for determining the pressure difference is arranged outside the heart in the thorax or outside the patient's body. In addition, it can be provided that the sensors are connected to radio equipment for transmitting measured values or to an electrical lead passing through the heart wall. The sensors can also be formed as passive surface wave sensors, in the form of a transponder, which can be queried by radio.

Since the pressure difference between the determined measured values of both pressure sensors is to be determined, these measured values can be transmitted firstly to a device for determining a pressure difference. Here, the values can be transmitted electrically or by means of radio transmission or by means of other transmission methods which are not physically bound. The device for determining a difference in pressure can then form a difference in an analogue or digital manner and can provide the pressure difference value or transmit it directly to a control unit of the device, in particular of the heart pump. The control unit of the device can also take on the pressure difference determination directly.

With use of an individual differential pressure sensor, which is acted on by both pressures to be compared, the forming of the difference of this kind of course is not necessary, or is performed directly by use of the corresponding differential pressure sensor principle.

The object is also achieved by a method for open-loop or closed-loop control of an assembly in a patient's body under consideration of a pressure measured in a patient's heart, wherein the pressure is measured in the form of a difference in pressure between the inside of the heart and the region outside the heart in the thorax and is used as a basis for the open-loop or closed-loop control.

In order to carry out the method, the above-described device can be used, in particular in conjunction with a control unit of an implanted device, for example a heart pump, a cardiac pacemaker or a defibrillator.

In addition, the solution relates to a computer program product comprising a computer program for open-loop or closed-loop control of an assembly in a patient's body under consideration of a pressure measured in the patient's heart, wherein the pressure is determined as a difference in pressure between the inside of the heart and the region outside the heart in the thorax and is used as a parameter in the computer program. A computer program of this kind is used for example to operate a microcontroller, which processes the detected measurement data. However, the program can also be operated as a sub-program or program module with operation of a control unit of a corresponding assembly, for example a heart pump.

In order to solve the problem, a controller for controlling a heart pump can additionally be provided, in which parameters, for example such as filling pressures of the heart on the basis of the difference in pressure between the pressure inside the heart and outside the heart in the thorax, determined by a device, are used and in which the delivery capacity of the pump is used as a manipulated variable.

In addition, the solution in conjunction with a cardiac pacemaker can also provide a controller for controlling a cardiac pacemaker, in which parameters, for example such as filling pressures of the heart on the basis of the difference in pressure between the inside of the heart and the space outside the heart in the thorax, determined by a device, are used and in which the delivery capacity of the heart is used as a target variable of the control.

The described solutions to the problem will be presented hereinafter in figures of a drawing and will be explained further below. In the drawing

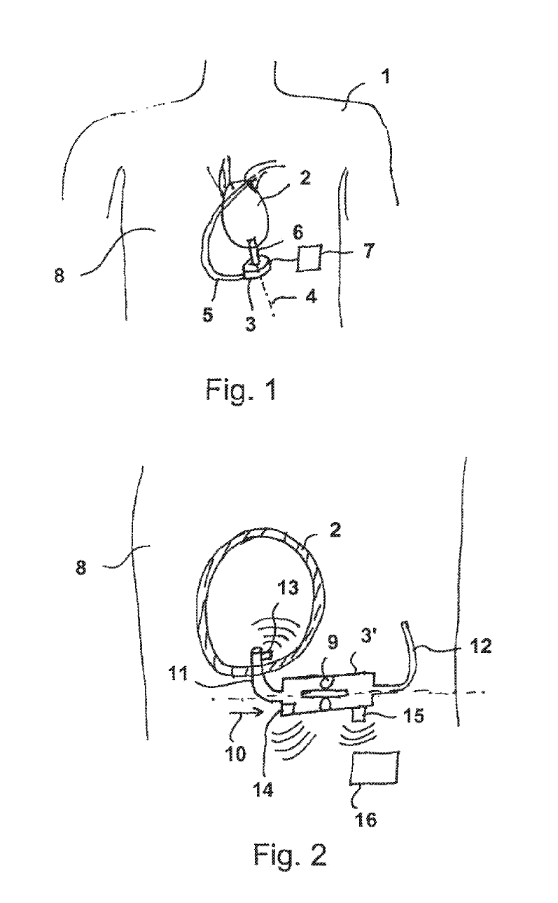

FIG. 1 shows a patient's body in a schematic illustration, showing a heart and a heart pump,

FIG. 2 shows a schematic illustration of a heart as a cavity with a heart pump connected thereto with sensors,

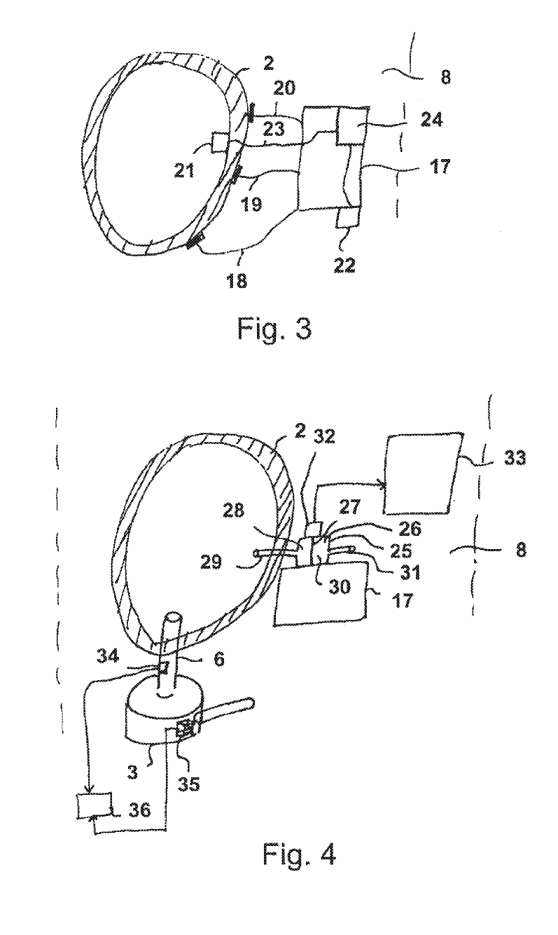

FIG. 3 schematically shows an illustration of a heart with two sensors, which are connected to a device,

FIG. 4 shows a schematic illustration of a heart with two possible arrangements of devices which each have sensors for measuring pressure,

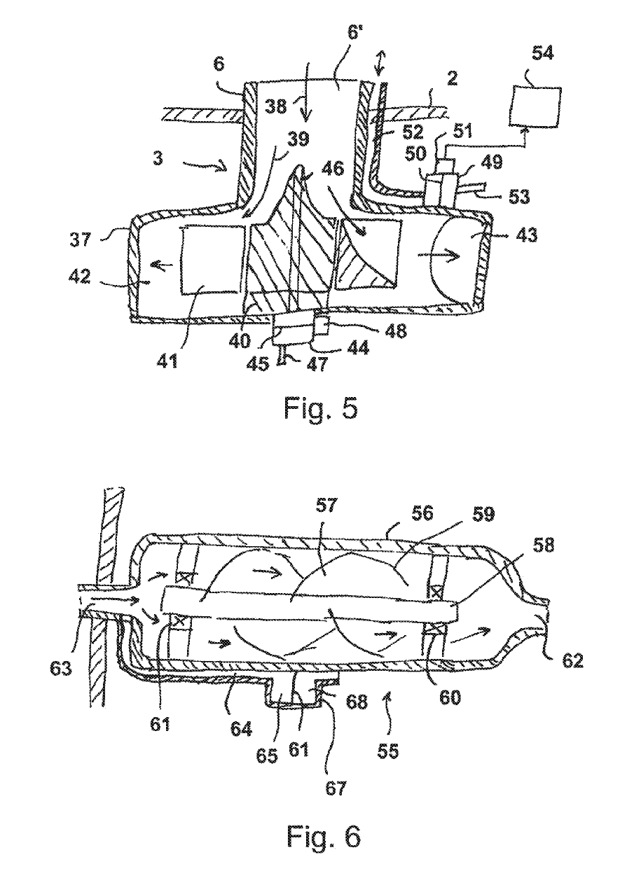

FIG. 5 shows the structure of an axial/radial pump with a differential pressure sensor, and

FIG. 6 shows the structure of an axial pump with a differential pressure sensor.

FIG. 1 schematically shows the body 1 of a patient, wherein the heart 2 is illustrated separately and schematically with an implanted heart pump 3.

The heart pump 3 has a rotor, which is rotatable about the axis 4 and in so doing conveys blood into a catheter 5. The pump 3 draws in blood from the interior of the heart, for example from an atrium or a ventricle, through an aspiration port 6, which passes through a wall of the heart 2, and conveys it via the cannula/the catheter 5 into a blood vessel.

A control unit 7 is additionally illustrated, which controls the pump 3, for example by means of electrical signals. The pump 3 is driven by an electric motor having windings and magnets. The motor can be a brushless electric motor, for example controlled by means of a pulse width modulation method.

The rotor of the pump 3 can be formed as a radial rotor, which axially accelerates inflowing fluid in a radial direction. The conveyed fluid/blood is then collected in a radially outer collection space and is conducted radially outwardly into the cannula 5 by means of the generated pressure.

In order to control the pump 3 by means of the control unit 7, it is important to take into consideration the pressure conditions in the heart 2 in comparison to the pressure prevailing in the thorax 8 outside the heart. The difference in pressure between the space inside the heart 2 and the thorax 8 is measured by a sensor device, which will be explained in greater detail further below.

FIG. 2 schematically shows, in a cross-section, the patient's heart 2, which is illustrated here merely as a cavity and is arranged in the thorax 8. A heart pump 3′ is provided, which is formed as an axially conveying pump with a rotor 9. For example, the pump conveys blood from inside the heart 2 in the direction of the arrow 10, thus draws it in from the heart by means of the aspiration cannula 11, and discharges it through the outflow cannula 12 in the direction of a blood vessel (not illustrated). In order to measure the difference in pressure between the inside of the heart 2 and the thorax, at least two sensors of a sensor device can be provided. A total of three sensors are illustrated in FIG. 2, wherein the sensors 13, 14 are intended as alternatives and each measure the pressure within the heart. Here, the sensor 13 is arranged directly inside the heart, whereas the sensor 14 within the pump is connected to the inside of the heart by the aspiration port 11 and measures the pressure inside the heart directly, aside from flow resistances. By contrast, the sensor 15 is arranged outside the pump 3′ in the thorax 8 and measures the pressure there.

The three sensors 13, 14, 15 are each equipped with radio devices or induction coils, which allow them to send measured values to a device 16 for determining the difference in pressure. The device 16 can be formed for example as a microcontroller and can be equipped with a receiver for receiving the measured data. A computer program, which runs on the microcontroller, determines the difference in pressure detected by the various sensors 13, 14, 15 of the sensor device. This can then be forwarded to a control unit of the pump 3′. The sensors 13, 14, 15 are directly arranged on and secured to the heart pump 3′ or to the aspiration port 11 of the pump. They can be secured here to the outside of the pump housing, as is the case for example with the sensor 15, but can also be secured within the pump chamber on the inside of the housing, as is the case with the sensor 14.

FIG. 3 shows a cardiac pacemaker 17 which is implanted in the thorax 8 of a patient. The cardiac pacemaker 17 is connected by means of various leads 18, 19, 20 to electrodes, which are applied directly against the wall of the heart 2. In order to take into consideration the transmural pressure, i.e. the difference in pressure between the inside of the heart 2 and the thorax 8, two sensors 21, 22 are provided, wherein the first sensor 21 is arranged inside the heart 2 and is connected to the cardiac pacemaker 17 by means of an electrical lead 23. The sensor 22 is directly secured externally to the housing of the cardiac pacemaker 17 and is used to measure the pressure in the thorax. Both sensors 21, 22 are connected by means of leads to a device 24 for determining a difference in pressure, which device is integrated in the cardiac pacemaker 17. The determined difference in pressure can thus be used within various algorithms of the cardiac pacemaker 17.

FIG. 4 schematically shows the heart 2 of a patient with two assemblies that can be used alternatively, which each allow a difference in pressure to be determined. In the right-hand part of the figure, a cardiac pacemaker 17 is shown merely schematically as a box. There is secured thereto a differential pressure sensor 25, which provides a separation membrane 27 in a measurement chamber 26. The first sub-chamber 28 on the side of the membrane 27 facing the heart 2 is connected to the inside of the heart by means of a cannula 29. The second sub-chamber 30, which is located on the side of the membrane 27 facing away from the heart 2, is connected to the inside of the thorax 8 by means of a cannula 31. The membrane position of the resilient, flexible membrane 27 directly indicates the difference in pressure between the inside of the heart 2 and the thorax. This is detected and evaluated by a sensor and microcontroller 32 and is fed as a pressure difference value directly to the control unit 33. This can act in turn on the cardiac pacemaker 17 and can be connected thereto.

A heart pump 3 is additionally shown in FIG. 4, which heart pump is formed as an axial/radial pump and has an aspiration port 6, which passes through the wall of the heart 2 and is fitted tightly in the opening of the heart wall. A first sensor 34 is arranged within the aspiration port 6 and is connected via the interior of the aspiration port 6 to the interior of the heart 2 and measures the absolute pressure prevailing there. In addition, a sensor 35 is arranged on the outside of the pump 3 and detects the absolute pressure in the surroundings of the pump 3, that is to say in the thorax. Both sensors 34, 35 are connected by radio or by means of an electrical lead to a device 36 for determining a difference in pressure, which device determines the difference in pressure between the inside of the heart and the thorax.

An axial/radial pump 3 is shown by way of example in greater detail in FIG. 5. The pump has a housing 37 with an aspiration port 6, which passes through the wall of the heart 2. An inflow channel/aspiration channel 6′ is formed in the aspiration port 6, through which blood is aspirated in the direction of the arrows 38, 39.

The pump motor has an inner stator 40, on which the rotor 41 can be mounted hydrostatically and/or magnetically and in the radial and axial direction. The pump motor can be formed as a brushless electric motor. Flow channels for the blood are integrated in the rotor and allow blood to flow in the axial direction and to be discharged in the radial direction by means of a centrifugal effect. An annular collection chamber 42 is formed in the housing 37 around the rotor 41, in which collection chamber the blood collects and flows in a peripheral direction to an outflow opening 43, from where it is moved on into an outflow cannula.

Two examples of a differential pressure measurement are illustrated on the basis of the pump in FIG. 5. A differential pressure sensor 44 having a membrane 45 is firstly illustrated, wherein the sensor on the one hand communicates by way of a channel 46 in the stator with the intake region of the pump in the aspiration port and on the other hand communicates with the pump exterior by way of a fluid channel 47. The difference in pressure is evaluated in an evaluation device 48 and is forwarded accordingly to an assembly.

The second differential pressure device, which is conceivable alternatively on a pump of this kind, has a differential pressure sensor 49 with a membrane 50 and an evaluation device 51. The sensor 49 is connected on one side of the membrane 50 to the inside of the heart by way of a channel 52, which runs in or on the housing 37 parallel to the aspiration channel 6′. The channel 52 can be formed by a curved tube placed externally on the housing 37 or by a bore in the wall of the housing 37 or also by a tube running on the inner wall of the housing 37.

The sensor 49 communicates on the other side of the membrane with the external surroundings of the pump housing 37 in the thorax by way of a cannula 53. The device 51 for determining a difference in pressure communicates with an assembly 54.

An axial pump 55 with a partially cylindrical housing 56 is illustrated in FIG. 6, in which housing a rotor 57 is rotatably mounted. The rotor 57 has a hub 58 with conveying elements 59. The hub 58 is rotatably mounted in two bearings 60, 61 and is usually driven by means of a motor or a flexible shaft, which is not illustrated in the figure. The flexible shaft usually protrudes through the outflow opening 62 of the housing 56. The inflow opening is denoted by 63.

A channel 64 is connected to the inflow opening 63 and runs externally on the wall of the housing 56 and leads to a first sub-chamber 65 on one side of the membrane 66 of a differential pressure sensor 67. The second sub-chamber 68 of the differential pressure sensor 67 is connected to the external surroundings of the pump housing 56 by way of an opening and/or a fluid channel. The fluid channel 64 can also run within the housing wall of the housing 56 or on the inside of the housing wall of the housing 56 between the inflow opening 63 and the differential pressure sensor 67.

By means of the described solutions, it is possible to determine the difference in pressure between the absolute pressure in a patient's heart and the thorax in a simple manner, wherein both the production and implantation are facilitated by means of the integration of the sensor device at least in part in an implantable assembly.

The actual filling pressure of the heart can be determined using the described sensor device without potential influences by breathing, “pressing” or pathological changes to the thoracic pressure. A more advantageous signal can therefore be measured using the same number of pressure sensors as before. This enables a quicker control of heart pumps than before, which control is more robust with respect to these interfering influences. This would lead to a consequent prevention of aspiration and, by means of the quicker adaptation to physical load, to an improved quality-of-life for the patient. Detection of a pathological change in the thoracic pressure (for example pericardial effusion, pericardial tamponade) also appears to be conceivable.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, or N including any one element alone or the one element in combination with one or more of the other elements which may also include, in combination, additional elements not listed.

1. A device for measuring pressure in a patient's heart, the device comprising a sensor device configured to detect a difference in pressure between a point inside the heart and a space that is outside the heart and inside a thorax of the patient.

2. The device according to claim 1, wherein the sensor device comprises: a channel passing through an outer wall of the heart, wherein the channel communicates both with a region lying outside the heart in the thorax and with a region lying inside the heart; and a differential pressure sensor that is connected both to the first region lying outside the heart and to the second region lying inside the heart.

3. The device according to claim 2, wherein the differential pressure sensor includes a device configured to close a passage opening in the heart wall of the heart.

4. The device according to claim 2, wherein the channel is formed in or on a blood pump.

5. The device according to claim 4, wherein the differential pressure sensor is connected to a region inside the pump or in an intake region thereof or a discharge region thereof on one hand, and to a region outside the pump housing and the blood vessel on the other hand.

6. The device according to claim 2, wherein the channel runs through an interior of a pump housing and is surrounded at least in part, in particular in portions, on all sides by the blood conveyed by the pump, wherein in particular it is provided that the pump aspirates blood in the axial direction and conveys it in the radial direction.

7. The device according to claim 2, further comprising a pump with a channel running on a housing wall of said pump separated from the pump chamber.

8. The device according to claim 1, wherein the sensor device has two absolute pressure sensors, the first of which is arranged inside the heart or is connected to the inside of the heart by way of a fluid channel and the second of which is arranged outside the heart in the thorax or is connected to a region outside the heart in the thorax by way of a fluid channel, and these are connected to a device for determining a difference in pressure.

9. The device according to claim 8, wherein the device for determining a difference in pressure is arranged outside the heart in the thorax or outside the patient's body.

10. The device according to claim 8, wherein the first sensor is connected to a radio device for transmitting measured values or to an electrical lead passing through the heart wall.

11. The device according to claim 1, wherein at least one sensor of the sensor device is formed as a surface wave filter, and/or wherein induction coils are provided for transmitting sensor data and/or the power supply of a sensor.

12. A method for open-loop or closed-loop control of an assembly in a patient's body based on a pressure detected in the patient's heart, the method comprising detecting the pressure as a difference in pressure between the inside of the heart and a region outside the heart in the thorax, wherein the pressure detected forms the basis of the open-loop or closed-loop control.

13. A computer readable medium comprising instructions executable with processor for open-loop or closed-loop control of an assembly in a patient's body based on a pressure detected in the patient's heart, wherein the instructions are executable to determine the pressure as a difference in pressure between the inside of the heart and a region outside the heart in the thorax wherein the pressure detected is used as a parameter in the executable instructions for open-loop or closed-loop control of the assembly.

14. A controller for controlling a heart pump or a cardiac pacemaker, wherein a sensor device is configured to determine a difference in pressure between the pressure inside the heart and outside the heart in the thorax, and the controller is configured to use the difference in pressure as a measurand and a delivery capacity of the pump as a manipulated variable if the controller is for controlling the heart pump or a delivery capacity of the heart as a target variable of the control if the controller is for controlling the cardiac pacemaker.

15. (canceled)
